(19) United States
(12) Patent Application Publication
(10) Pub. No.: US 2002/0165483 A1
(43) Pub. Date: Nov. 7, 2002

(54) Gas assisted spray applicator

(76) Inventors: Curtis Miller, Inver Grove Heights, MN (US); Jon Hoogenakker, Inver Grove Heights, MN (US); Alan Lonnemann, Plymouth, MN (US)

Correspondence Address: John M. Vasuta, 7570 Hudson Park Drive, Hudson, OH, 44236, US

(21) Appl. No.: 10/008,415

(22) Filed: Nov. 13, 2001

Related U.S. Application Data
(60) Provisional application No. 60/246,921, filed on Nov. 10, 2000.

(51) Int. Cl. A61M 37/00

(52) U.S. Cl. 604/82; 606/213; 604/191

(57) ABSTRACT

A medical fluid delivery system for reactive fluids such as tissue adhesive fluids including fibrin glues whereby reactive fluids in separate syringes are expelled from the system while a separate compressed gas is released around, adjacent to, or proximate the expelled reactive fluids such that the fluids and gas mix and are propelled onto a site.

### PRIORITY

This patent application claims priority to currently pending (through Nov. 13, 2001 due to weekends and government holidays) provisional patent application no. 60/246,921 filed on Nov. 10, 2000.

### BACKGROUND OF THE INVENTION

1. Technical Field

The invention relates generally to medical fluid delivery systems. More particularly, the invention relates to medical fluid delivery systems for the application of two liquids in surgery.

2. Background Information

In the medical field, fluids are sprayed, swabbed, poured, squirted, applied, or otherwise provided onto a surgical site, an abrasion, or other desired area. Often these fluids are reactive, that is two separate fluids react when mixed. Some examples of medical applications that use such reactive fluids are: hemostasis or bleeding control such as after surgery or for hemophiliacs, sealing such as on burns, hole closure such as puncture wounds or to close lung holes, gluing or adhesion, and adhesion prevention to prohibit two surfaces from growing together where it is not desirable.

As an example, the area of hemostasis or bleeding control often uses fibrin glue to stop bleeding such as after surgery. The basis of this is that it has been known for many years that two principal components within blood are responsible for coagulation, namely fibrinogen and thrombin, and that separation of such components prior to contact with oxygen allows for later recombination thereof and thus some form of blood coagulation at the point of recombination, or on the nearest surface thereto. Obviously, control of this process is valuable as medical and veterinary professionals may then control bleeding (hemostasis) such as during or after surgery.

These tissue adhesives have been the focus of patents and technology back to at least 1950 as indicated by U.S. Pat. No. 2,533,004 which disclosed various methods for making fibrin clots using different concentrations of fibrinogen solution in conjunction with a thrombin solution. In recent years, the study of tissue adhesives has become very popular. Various of these tissue adhesives, often referred to as fibrin glues, have been developed and are currently pending before or recently approved by the U.S. Food and Drug Administration and will be available for surgical and other medical uses should, or when, any are approved. Some of these are presently in use outside of the U.S. or inside the U.S. in veterinary applications.

Depending upon the percentages of fibrin and thrombin in each respective solution, as well as the other components found in each solution, various factors are critical to the mixing and application of the components. These factors include viscosity of the initial fibrinogen and thrombin solutions and the final mixed solution, diameters or sizes of the fluid passages and mixing compartment, mixing rate of the fibrinogen and thrombin solutions particularly in comparison to ejection rate if mixing occurs within the system, and others.

Numerous tissue adhesive applicators have been developed such as those described in U.S. Pat. Nos. 4,040,420 (Speer), 4,359,049 (Redl), 4,733,666 (Eibl), 4,826,048 (Skorka), 4,874,368 (Miller), 4,902,281 (Avoy), 4,978,336 (Capozzi), 5,116,315 (Capozzi), 5,368,563 (Lonnemann), and 5,474,540(Miller). A variety of types of these applicators exist including internal swirl or mixing chamber applicators, and external combining applicators such as external swirl applicators and external spray or stream overlapping applicators.

Of these, the applicators in U.S. Pat. Nos. 4,826,048; 4,978,336; 4,979,942; 5,116,315; are swirl or other pre-ejection mixing applicators where mixing is performed by squirting or otherwise forcing both fluids into a swirl or other mixing chamber where the fluids mix to some degree based upon turbulence in the swirl chamber and the material properties, and are thereafter ejected from the applicator. In short, the mixing occurs inside of the applicator and thus time is critical as it must be ejected prior to coagulation. In some applications such as where one or more of the solutions or fluids is thick or highly viscous, internal swirling results in only marginal or partial mixing; while in other applications such as where all of the solutions or fluids are thin or not-highly viscous, substantial and effective mixing occurs.

In contrast, the applicators in U.S. Pat. Nos. 4,040,420; 4,874,368; 4,902,281; 5,368,563; and 5,474,540 are external combining applicators. External combining is the process of bringing the two solutions into contact with one another at the point of use for functional tissue adhesive creation. External combining eliminates the premature mixing problems. However, with many external combining applicators thorough mixing of the solutions does not occur and instead only adjacent portions of the solutions mix or combine while large percentages remain unmixed or uncombined. This results in inefficient and somewhat uncontrolled coagulation.

Various applicators exist to perform such external combining with varying degrees of success. Just as in internal mixing, it has been found in external combining that its success vastly improves when thin or not-highly viscous solutions are used, while thick or highly-viscous solutions perform poorly when externally combined since more than mere fluid interaction is needed.

The following are a few examples of external mixing. The '563 patent owned by Micromedics Inc. is an external swirling patent where each of the fluids is sprayed in a swirl pattern that overlaps the other fluid's swirl pattern resulting in fluid mixing. In contrast, the '368 and '540 applicators eject streams that intersect whereby the fluids combine.

Currently, many of the current fibrin glue solutions being proposed to or recently approved by the U.S. Food and Drug Administration contain at least one highly viscous component and are thus not readily nor effectively combined in an external manner as the highly viscous solution does not combine or mix with the other solution whether it be thin (not-highly viscous) or thick and highly-viscous. In addition, these highly viscous fluids often are difficult at best to force, eject, expel or otherwise push out of a syringe or other storage chamber. This is particularly true when manually actuating the syringes, and particularly in designs with small diameter passages or channels which is very typical.

For these and other reasons, it is desirable to develop a system, device and/or method of thoroughly mixing the reactive fluids or liquids of any viscosity. This new design must or should use standard available methods (i.e. syringes) for preparation and application of materials, produce a spray output for improved mixing and control of application, allow for various sizes and ratios of syringes, allow for various tip configurations for different medical procedures, provide sterile air and a fully sterile apparatus, be completely portable and self-contained, and avoid clogging. As a result, a new design is needed for the application of two reactive liquids, such as the components of some fibrin glues, where thorough mixing of liquids regardless of viscosity occurs. It is contemplated that such a system will have a wide variety of uses in the medical and other industries.

### SUMMARY OF THE INVENTION

The invention is a novel, useful and nonobvious medical fluid delivery system for mixing and delivering two or more reactive fluids such as tissue adhesives including fibrin glues. The medical fluid delivery system includes an applicator through which both (a) two components from separate syringes or storage facilities are expelled, and (b) a separate compressed gas is released around, adjacent to, or otherwise proximate with the expelled components, resulting in a thorough mixing of the components and compressed gas, plus propulsion of the mixed components and compressed gas to a surgical or like site.

### BRIEF DESCRIPTION OF THE DRAWINGS

The preferred embodiments of the invention, illustrative of the best mode in which applicant has contemplated applying the principles, are set forth in the following description and are shown in the drawings and are particularly and distinctly pointed out and set forth in the appended claims.

FIG. 1 is a digital image of the gas assisted spray applicator of a first embodiment the present invention;

FIG. 2 is a digital image of the gas assisted spray applicator of FIG. 1 in a disassembled or exploded view;

FIG. 3 is a drawing of the top view of the mixing tip portion of the gas assisted spray applicator of FIGS. 1-2;

FIG. 4 is a drawing of a side view of the mixing tip and syringe manifold portions of the gas assisted spray applicator of FIGS. 1-3;

FIG. 5 is a drawing of the bottom view of the mixing tip and syringe manifold portions of the gas assisted spray applicator of FIG. 4;

FIG. 6 is a drawing of an end view of the mixing tip portion of the gas assisted spray applicator of FIG. 3;

FIG. 7 is a drawing of an end view of the mixing tip portion of the gas assisted spray applicator of FIG. 4;

FIG. 8 is a drawing of the top view of the mixing tip portion of the gas assisted spray applicator of FIGS. 1-8 with the internal structure shown in hidden lines;

FIG. 9 is a drawing of a side view of the mixing tip portion of the gas assisted spray applicator of FIGS. 1-8 with the internal structure shown in hidden lines;

FIG. 9A is a drawing of the same side view as FIG. 9 except taken in cross section;

FIG. 10 is a drawing of the top view of the mixing tip similar to FIG. 3;

FIG. 11 is a drawing of the bottom view of the syringe manifold portion of the gas assisted spray applicator of FIGS. 1-10;

FIG. 12 is an enlarged view of FIG. 11 showing the lumens extending from the end portion of the manifold where the lumens are not the same length;

FIG. 13 is a drawing of a second embodiment of the entire gas assisted spray applicator where the compressed gas is an in wall system (not shown);

FIG. 14 is a drawing of a third embodiment of the entire gas assisted spray applicator where the compressed gas and reactive fluid subsystems are separate;

FIG. 15 is a drawing of the first embodiment of the entire gas assisted spray applicator where the compressed gas and reactive fluid subsystems are combined as is shown in FIGS. 1-2;

FIG. 16 is an enlarged drawing of the second embodiment of the entire gas assisted spray applicator attached to a portable compressed gas source; and

FIG. 17 is a drawing of the second embodiment of the entire gas assisted spray applicator as shown in FIG. 13 where the compressed gas is an in wall system as shown.

Similar numerals refer to similar parts throughout the drawings.

### DESCRIPTION OF THE PREFERRED EMBODIMENTS

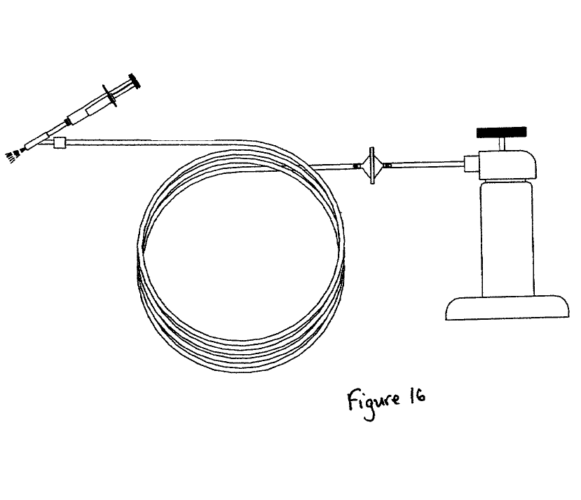

The invention is a medical fluid delivery system 10 which expels two components, at least one of which is often reactive, from separate syringes or storage facilities and then releases a pressurized fluid such as compressed gas around, adjacent to, or proximate the reactive components causing the thorough mixing of the reactive components and compressed gas as well as propulsion thereof in a fluid stream onto a surgical or like site. The medical fluid delivery system 10 is shown in FIGS. 13-17 in several embodiments as a gas assisted fibrin glue spray applicator, although the system 10 may take other designs including those described below. The system 10 may be used to expel, mix and propel other reactive components of any viscosity, including highly viscous fluids such as some fibrin glue components, with the assistance of a compressed gas.

In the embodiment shown in FIG. 15, the system 10 includes multiple syringes 12 and 14 (hidden in FIG. 15), a syringe holder or link 16, a plunger clip 18, check valves 20 and 22 (also hidden), an applicator 24, gas conduit or passage 26, a compressed gas source 28, a regulation valve 30, and a filter 32. All of which once coupled together results in system 10 for application of reactive fluids of any viscosity, including highly viscous fluids, which are expelled from syringes 12 and 14 at the end face of applicator 24, whereby the fluids are thoroughly mixed together by the release of compressed gas, around, adjacent to or otherwise proximate the reactive fluids expelled from applicator 24. In addition to thorough mixing, the system 10 via the release of the compressed gas around, adjacent to or proximate the expelled reactive fluids also functions to simultaneously propel the mixture of compressed gas and reactive fluids onto a selected surgical or like site.

Syringes 12 and 14 are typical surgical or medical syringe designs as are well known in the medical profession. The syringe design is not critical and as such the syringes may be of any design known in the art so long as some movable body provides compression to any fluid within the syringe so as to force the fluid out of an exit within the syringe when so desired. The syringes may be the same size, or of a different size, and also of the same or different volumes, and the syringes may even be other forms of fluid storage chambers.

Each syringe basically includes a cylinder and a plunger, the cylinder being defined by an elongated body having a hollow cylindrical cavity therein for receiving the plunger. The cavity is completely open at one end with a connector flange therearound while having a base wall at the other end with an exit port therein. The base wall may be either perpendicular to a central axis of the cavity or it may be oblique so as to define a funnel shaped base. In the embodiment shown, the exit port is within a receiver that extends outward from the base wall whereby in one embodiment, this receiver is a luer-type receiver or other compression or tapered fitting receiver as are well known in the medical art for receiving luer-type connectors.

In the embodiment shown, the plunger includes an elongated body with a head on one end thereof as is known in the art. In one embodiment as is shown, this head includes a tapered face and a cylindrical body with a seal therein as is also known in the art. The elongated body preferably also has a flange or end plate against which pressure is exerted to selectively move the plunger within the cylinder to squirt the fluid within the cavity out the exit port. The flange also provides a surface against which to lock a plunger link as described above.

The syringe holder or link 16 includes a pair of indentations of a generally semi-cylindrical nature with a land therebetween. The body has a front end and a back end whereby each of the indentations includes a slot therein approximate the back end of the body. The slot being for receiving one of the connector flanges when one of the syringes is seated within the respective indentation.

In the embodiment of system 10 shown in FIG. 15, within the land are a pair of holes through which the compressed gas source 28 may be attached to the link 16, and an additional hole through which the regulation valve 30 may extend thereby allowing for ease of use during plunger actuation as is describe below in more detail. In the embodiment in FIG. 13 the source is an in-wall compressed gas system so the hose connects directly into it so there is no need to connect anything to the link. As to the embodiment in FIG. 14, the compressed gas source and the link are separate.

Alternatively, we may use an oval shape plate with two holes that the syringes go through. Fingers grip the syringes as they go through the holes.

An optional plunger clip 18 is available for linking the plungers on syringes 12 and 14 so as to cause simultaneous actuation of the plungers and thus accurate combining of the reactive fluids at the desired rates. The plunger clip is a body which includes a pair of thin slots sized and shaped to receive the flange or end plate of the plunger. The combination of the link 16 and the clip 18 allow the entire assembly 10 to be held and actuated in one hand.

Check valves 20 and 22 are optionally located between the applicator 24 and each of the syringes 12 and 14 and include a one way flow passage therein. These check valves function to prevent material from backing up into the syringe such as is likely where back pressure is present.

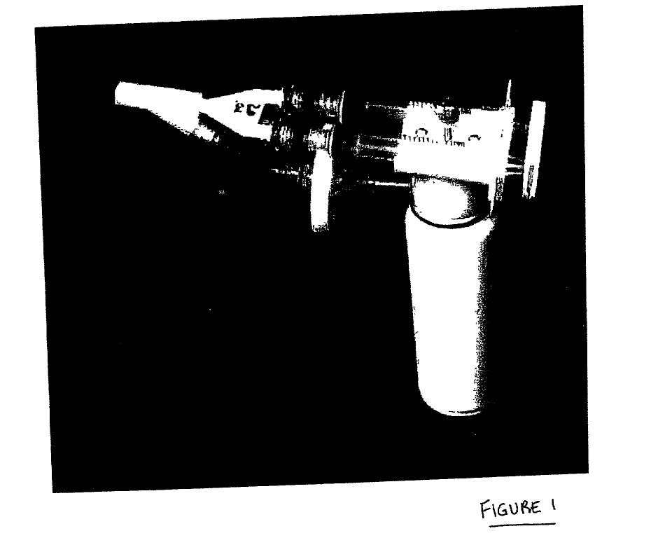

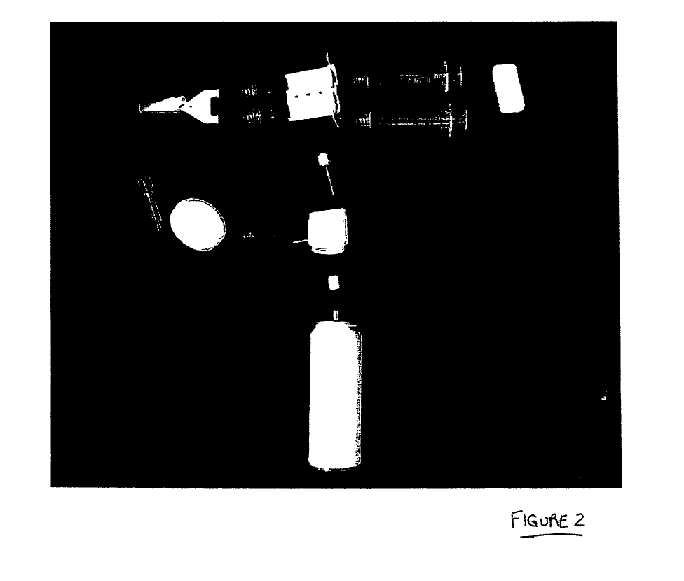

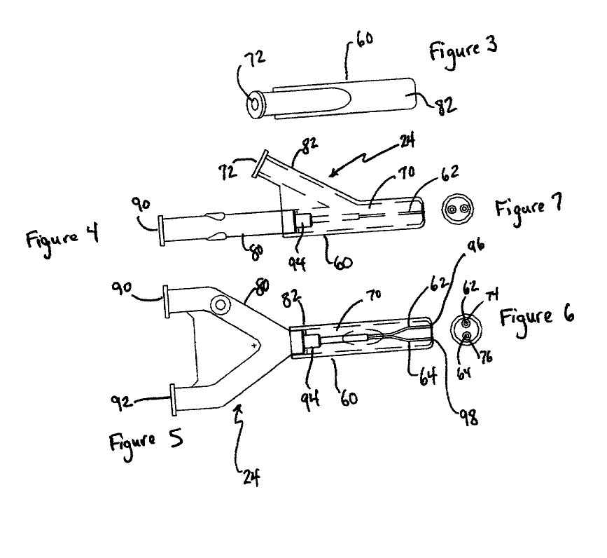

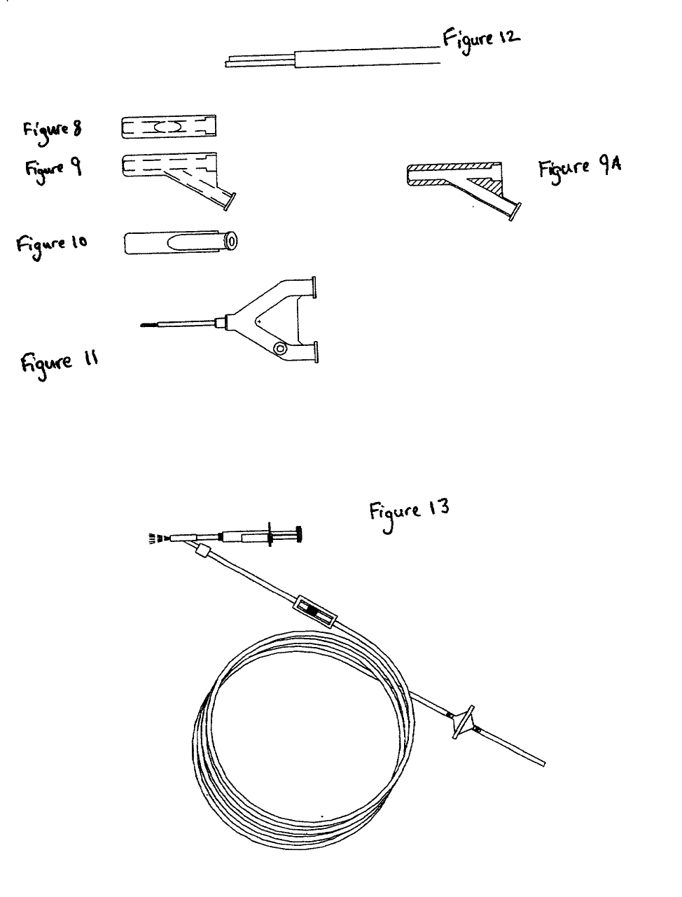

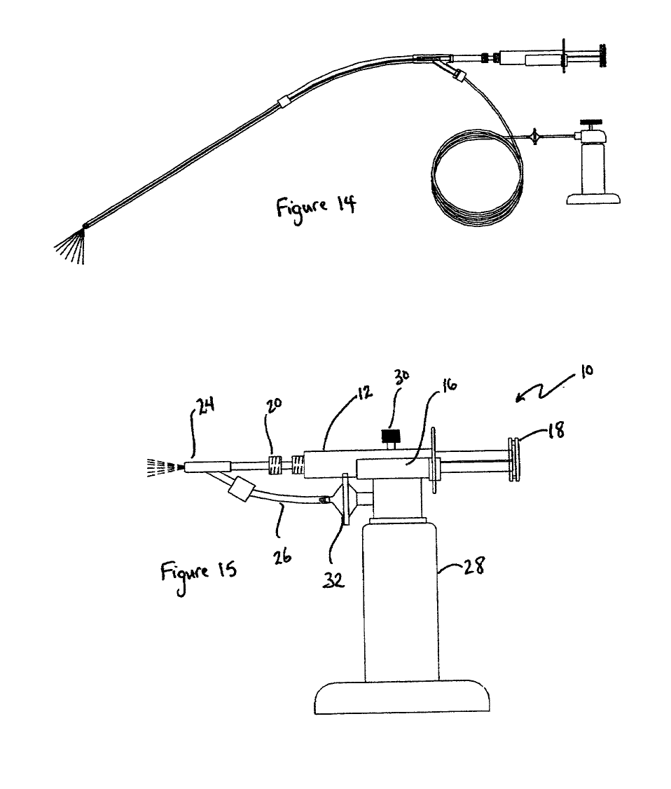

Applicator 24 is any device for expelling fluids from multiple syringes while also providing a means for releasing compressed gas around, adjacent to or proximate the outlets where the fluids from the multiple syringes are being expelled. In the embodiment shown in FIGS. 1-17, applicator 24 is a shell 60 with multiple or fluid passageways lumens 62 and 64 therethrough each having an inlet, a continuous passageway and an outlet. The shell further includes a hollow chamber 70 with an compressed gas inlet 72 as well as a means for providing compressed gas around, adjacent to or proximate the outlets of the multiple fluid passages.

In more detail as to the embodiments shown, two lumens 62 and 64 are provided extending through the applicator 24. Applicator 24 also includes the compressed gas inlet 72, the compressed gas chamber 70 and compressed gas outlets 74 and 76. The applicator 24 may be one integral body, or as shown in the Figures may be a two piece unit including a manifold 80 and a mixing tip 82.

In even more detail as to the embodiment shown in the Figures, manifold 80 includes inlets 90 and 92 which are fluidly connected to lumens 62 and 64, where syringes 12 and 14 are attachable thereto such that the fluids contained therein may be expelled from the syringes and flow directly into lumens 62 and 64 at the base of inlets 90 and 92. The manifold may be of any shape, but in this case is “Y” shaped so as to bring the lumens 62 and 64 closer together for delivery to the site. The lumens 62 and 64 extend from inlets 90 and 92 to end 94 of the manifold, and in this embodiment substantially extending therefrom.

As the lumens extend from the manifold body, the lumens may remain adjacent one another, or may separate slightly, depending upon the design and needs of the mixing tip. In the drawings, the lumens branch slightly apart to a desired separation for expulsion to the site. This separation keeps the reactive components away from each other at the end of the lumens. As a result, the fluid expelled from a syringe travels in through the respective inlet 90 or 92 and corresponding lumens 62 and 64, and is then expelled from the ends thereof at exit ports 96 or 98.

In the embodiment shown, mixing tip 82 includes the compressed gas inlet 72, the compressed gas chamber 70 and compressed gas outlets 74 and 76. The lumens travel in a sealed manner through chamber 70 which in the displayed embodiment is of a “y” shape. The exit ports 96 and 98 are smaller than, align with and extend through gas outlets 74 and 76 when the mixing tip is attached over the manifold 80.

The exit ports 96 and 98 may be flush with the end of the mixing tip, or alternatively may extend therefrom as a tube, and furthermore these exit ports may end at the same location, or one may extend further than the other as this helps further prevent polymerization of the reactive fluids at this point.

The mixing tube also includes compressed gas chamber 70 with a compressed gas inlet 72. The chamber 70 receives compressed gas from compressed gas conduit or passage 26. The fluids in the lumens or tubes however remain separate from each other and the released compressed gas while in the chamber 70.

In the preferred embodiment, outlets 74 and 76 surround or are around the exit ports 96 and 98. This allows the released compressed gas an escape route from the chamber 70 whereby the compressed gas rushes out of the outlets due to the lower atmospheric pressure outside of the tip than in the chamber 70 which is pressurized by the compressed gas. The compressed gas is still in a pressurized format in the chamber 66 as received from the compressed gas source 28 and thus it acts as a propellant whereby it propels itself and the other fluids adjacent or nearby. This fluid rush propels the reactive fluids expelled from the exit ports 96 and 98 whereby the compressed gas and reactive fluids are all propelled toward a surgical site. A vortex or suction-like result may occur.

In addition, during the fluid rush, the stream of compressed gas atomizes the materials including the reactive fluids. As a result, much smaller droplets of thoroughly mixed reactive fluids are sprayed. Overall, a better mixing than that currently offered by either internal or external sprayers is provided.

Source 28 is, in one preferred embodiment, a portable and disposable compressed gas cylinder as is shown in the FIGS. 14-16; however it is contemplated and preferred in many applications or environments that it could also be pressurized fluid or air from a larger system such as an in-wall system as is shown in FIG. 13. A gas conduit 26, such as a tube, provides fluid connection between the source 28 and the inlet 72 to the chamber 70.

The pressurized fluid or compressed gas may be freon, carbon dioxide, nitrogen, air or some other gas fit for surgical purposes. The gas may often be sterile when emitted or rendered sterile prior to emission by either radiation (gamma or the like) sterilization of the entire device 10 or by filtering of the gas via a sterile filter 32 between the source 28 and the gas conduit 26.

The source 28 is attachable to the link 16 via fasteners as is shown in the Figures. In addition, regulation valve 30 (FIG. 15) as attached to the source 28 may be positioned within the link 16 to provide for one hand actuation of the compressed gas via the regulation valve and the expulsion of the fluids at the plungers and clip. This is clearly shown in the Figures. This regulation and/or shutoff valve in various embodiments may be positioned within applicator such as on the link, or at the source whether a portable canister or an in-wall system with large pressurized tanks, or within the pressurized lines connecting the in-wall tanks with the applicator.

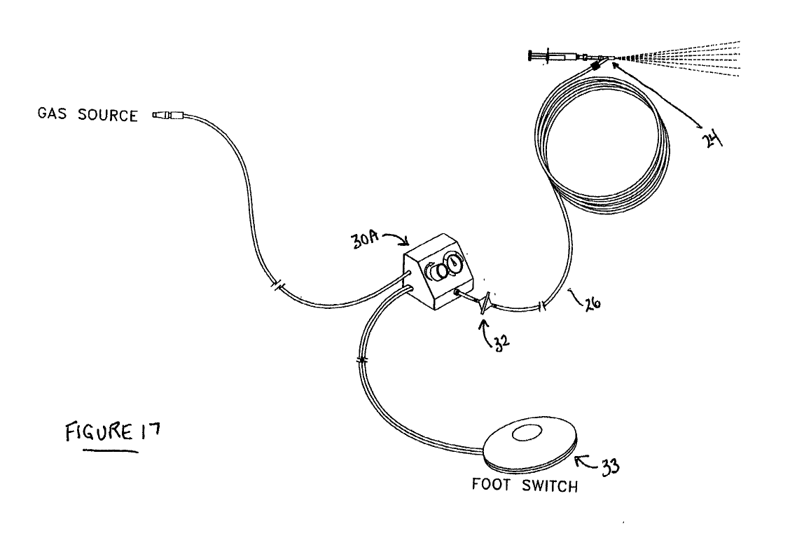

One embodiment of the in-wall system is shown in FIG. 17 to include multiple syringes 12 and 14, a syringe holder or link 16, a plunger clip 18, check valves 20 and 22, an applicator 24, a regulator system 30A, a foot switch 33, an elongated gas conduit 26 with a filter 32 therein extending from the regulator system to the applicator, another elongated gas conduit extending from the regulator system to a gas source, and a signal carrier whether electronic, optical or pneumatic connecting the foot switch to the regulator system.

In operation, the device or system 10 is used as follows. The surgical site may optionally need to be dried, whereby the surgeon or other medical personnel picks up the system 10 and starts the flow of compressed gas by opening the regulator 30 thereby causing the flow through the passage 26 and inlet 72, into chamber 60, and out outlets 74 and 76 where the compressed gas is blown onto the site. At any time this compressed gas flow may be stopped by shutting off the regulator valve 30.

When it is desired to deliver the reactive fluids to the site, the surgeon or other medical personnel picks up the system 10 and starts the flow of compressed gas by opening the regulator 30 thereby causing the flow through the passage 26 and inlet 72, into chamber 60, and out outlets 74 and 76 where the compressed gas is blown onto the site. The plungers of each syringe are actuated causing the reactive fluids therein to exit the respective syringe via its exit port, pass through the lumens 62 or 64, respectively, to exit ports 96 and 98 respectively, where the reactive fluids are expelled from the system 10. The reactive fluids are instantly expressed into the gas stream of compressed gas released from surrounding outlets 74 and 76 whereby the turbulence causes the reactive fluids to mix while simultaneously being sprayed onto the site.

After sufficient reactive fluids have been applied, the surgeon stops actuating the plungers on the syringes thus stopping the reactive fluids from being provided. Thereafter, the surgeon shuts off the regulator valve 30 thus stopping the release of compressed gas.

In any case, prior to expulsion from the system 10, the reactive fluids have remained separate and thus avoided any polymerization or reaction. Once expelled, the reactive fluids will come into contact and thereby polymerize or react to form a different substance such as fibrin glue. To avoid premature polymerization, in certain cases it is desirable to have the outlets not end at the same location so as to prohibit or reduce the chances of the reactive fluid from one outlet contaminating the other outlet.

It is also contemplated that the valve operation, both turning on and off, may be handled by a surgical or even scrub nurse in the vicinity of the surgeon. The compressed gas would thus be already on when the surgeon received the device 10 for reactive fluid application. This ease of handling is a result of the easy to use clip 18 that attaches the syringes for common actuation (which may be in 1:1 ratios, or other ratios as determined by syringe size, etc.) as well as the easy to use and efficiently located regulator valve 30 within the link 16.

Accordingly, the gas assisted spray applicator provides an effective, safe, inexpensive, and efficient device which achieves all the enumerated objectives, provides for eliminating difficulties encountered with prior devices, and solves problems and obtains new results in the art.

In the foregoing description, certain terms have been used for brevity, clearness and understanding; but no unnecessary limitations are to be implied therefrom beyond the requirement of the prior art, because such terms are used for descriptive purposes and are intended to be broadly construed.

Moreover, the description and illustration of the invention is by way of example, and the scope of the invention is not limited to the exact details shown or described.

Having now described the features, discoveries and principles of the invention, the manner in which the improved adhesive applicator is constructed and used, the characteristics of the construction, and the advantageous, new and useful results obtained; the new and useful structures, devices, elements, arrangements, parts and combinations, are set forth in the appended claims.

1. An adhesive applicator comprising:
a first syringe for storing a first solution;
a second syringe for storing a second solution;
a compressed gas source for providing compressed gas; and
a tip fluidly connected to both the first and second syringes for selectively receiving the first and second solutions in a separated manner and providing an expulsion port for each solution through which the solutions separately exit, the tip further being connected to the compressed gas source and providing for release of compressed gas around, adjacent to or proximate the expulsion ports such that the compressed gas when released mixes and propels the solutions and the compressed gas to a surgical site.

2. A mixing tip for attachment to a manifold for applying adhesive from attached syringes, the manifold including a plurality of syringe holders for receiving syringes and a plurality of corresponding lumens fluidly connected to the syringes and extending outward from the manifold in a manner such that the lumens expel fluids substantially adjacent to one another that mix thereafter, the mixing tip comprising a mixing body having a mixing chamber therein for selectively receiving the first and second solutions in a separated manner via the lumens and providing an expulsion port for each lumen through which the solutions separately exit, the tip further being connectable to a compressed gas source and providing for release of compressed gas around, adjacent to or proximate the expulsion ports such that the compressed gas when released mixes and propels the solutions and the compressed gas to a surgical site.
